SEQUENCE LISTING (11 sequences)

SEQ ID NO: 1 - protein, 20 aa, Mus sp.
CAGEIAMQANNHKCGVGVAY

SEQ ID NO: 2 - protein, 20 aa, Homo sapiens
CAGEIAMQANNHKCGVGVAY

SEQ ID NO: 3 - protein, 20 aa, Sus sp.
CAGEIAMQANNHKCGVGVAY

SEQ ID NO: 4 - protein, 20 aa, Rattus sp.
CAGEIAMQANNHKCGVGVAY

SEQ ID NO: 5 - protein, 20 aa, Aplysia sp.
CAGEVAIQANNHKCRVGIAY

SEQ ID NO: 6 - protein, 20 aa, Mus sp.
CAGEVAASANNSYCIVRIAY

SEQ ID NO: 7 - protein, 20 aa, Mus sp.
CAGEVAAVANNGVCGVGVAY

SEQ ID NO: 8 - protein, 20 aa, Cepeae sp.
CAGEVAAEANNTYCTIGIAP

SEQ ID NO: 9 - protein, 20 aa, Artificial Sequence
  Description of Artificial Sequence Synthetic peptide
CAGEIAMQANDHKCGVGVAY

SEQ ID NO: 10 - protein, 20 aa, Saccharomyces sp.
CAGEIAMQANNNFCGVGVAY

SEQ ID NO: 11 - protein, 20 aa, Bacillus subtilis
VAGTVAAQANNNSIGVLVAP

(19) United States
(12) Patent Application Publication
(10) Pub. No.: US 2008/0250516 A1
(43) Pub. Date: Oct. 9, 2008

(54) Prohormone Convertase 1 Mutation Associated with Obesity

(75) Inventors: Nicholas Gekakis, San Diego, CA (US); David Lloyd, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 10/591,885

(22) PCT Filed: Mar. 7, 2005
(86) PCT No.: PCT/US05/07300

§ 371 Date: Sep. 6, 2006

Related U.S. Application Data
(60) Provisional application No. 60/550,611, filed on Mar. 6, 2004.

(51) Int. Cl. C12N 15/55; C12N 9/48; A01K 67/027

(52) U.S. Cl. 800/18; 435/212; 536/23.2; 800/14

(57) ABSTRACT

The invention provides novel prohormone convertase 1 mutants that have at least partially defective enzymatic activities. These mutant PC1 enzymes are defective in their autocatalytic activities, but appear to have normal ability in processing proGHRH to its mature form. Also provided in the invention are non-human animals that harbor such a mutant PC1 gene. These non-human animals are obese due to the mutant PC1 gene.

### FIELD OF THE INVENTION

The present invention relates to enzymes responsible for processing prohormones and neuropeptides. More particularly, the present invention relates to prohormone convertase 1, novel properties of its mutants, and its role in regulation of energy homeostasis.

### BACKGROUND OF THE INVENTION

Prohormone convertase 1 (PC1) is a member of the mammalian family of endoproteases which are responsible for cleavage of many prohormones to generate bioactive hormones, similar to bacterial subtilisin and yeast Kex2 endoprotease. PC1 is found in neural and endocrine cells equipped with a regulatory-secretory pathway. The important role of PC1 in human hormonal biosynthesis has been elucidated by studies of a patient with defective PC1. See, e.g., Jackson et al., Nat. Genetics 16: 303-306, 1997. The patient lacking PC1 had severe childhood-onset obesity, postprandial hypoglycemia, infertility, and low levels of ACTH and cortisol with elevated levels of proopiomelanocortin (POMC). One product of POMC processing, α-MSH, is known to play a role in energy homeostasis. It is also known that PC1 is activated by autoproteolytic processing (see, e.g., Goodman and Gorman, Biochem. Biophy. Res. Comm. 201: 795-804, 1994). In addition, in vivo regulation of PC1 mRNA by thyroid status was shown in rat pituitary.

There is a need in the art for better understanding of prohormone processing and energy homeostasis, which would lead to improved means for preventing, diagnosing and treating obesity. The present invention fulfills this and other needs that will be apparent upon complete review of this disclosure.

### SUMMARY OF THE INVENTION

In one aspect, the present invention provides isolated mutant prohormone convertase 1 (PC1) polypeptides. Some of these polypeptides contain a missense mutation at residue Asn222 in the catalytic domain. In some embodiments, the missense mutation is substitution of Asn222 with an acidic amino acid residue. For example, the acidic amino acid residue can be aspartic acid.

In another aspect, the invention provides isolated polynucleotides encoding mutant PC1 polypeptides of the invention. The mutant PC1 polypeptides contain a missense mutation at residue Asn222 in the catalytic domain, e.g., a N222D substitution. In some embodiments, the PC1-encoding polynucleotides are obtained from a mammalian species. For example, they can be a human PC1 polynucleotide having accession number NM_{—}000439 or X64810, or a mouse PC1 polynucleotide having accession number NM_{—}013628, M69196, or M58589.

In a related aspect, the invention provides non-human animals which harbor a mutant prohormone convertase 1 (PC1) gene. The mutant PC1 gene encodes a polypeptide that contains a missense mutation at residue Asn222 in the catalytic domain. In some embodiments, the non-human animal is a mouse, and the mutant PC1 gene is mouse PC1 gene. In some other embodiments, the non-human animal is a rat, and the mutant PC1 gene is rat PC1 gene.

In another aspect, the present invention provides non-human animals that harbor in their genomes a DNA sequence encoding a mutant PC1 polypeptide. The encoded PC1 polypeptide is defective in its autocatalytic activity relative to that of the wildtype form of the PC1 polypeptide. In some of the animals, the encoded mutant PC1 polypeptide contains a missense mutation at residue N222, e.g., a N222D substitution.

### BRIEF DESCRIPTION OF THE DRAWINGS

FIGS. 1A-1B show the size and sequence differences between the PC1^{N222D} mutant mouse and a wildtype littermate.

FIG. 2 shows amino acid sequence comparison of PC1 from several mammalian species and other subtilisin/kexin protease family members.

FIGS. 3A-3B show weight gain in the PC1^{N222D} mutant mice as compared to a wildtype mouse.

FIGS. 4A-4C show weight and body composition differences among the PC1^{N222D} mutant mice and their wildtype littermate.

FIGS. 5A-5B show comparisons of food intake and activity among the PC1^{N222D} mutant mice and their wildtype littermate.

FIGS. 6A-6C show elevated insulin/proinsulin levels and glucose intolerance in PC1^{N222D} mice.

FIG. 7 shows defective auto-processing of PC1 in tissues obtained from a PC1^{N222D} mutant mouse.

### DETAILED DESCRIPTION

### I. Overview

The present invention is predicated in part on the discoveries by the present inventors that a single amino acid substitution in the prohormone convertase 1 gene can lead to important biochemical and physiological consequences. It was found that mice which contain an Asn to Asp substitution in a peripheral loop in the catalytic domain of PC1 are obese. It was also found that the autocatalytic activity of PC1 is perturbed as a result of this mutation. In addition, there does not appear to be any difference in food intake between the PC1 mutant mice and wildtype mice. Instead, there is a difference in activity, and by extension in energy expenditure, between the mutant and wildtype mice, with the PC1 mutant mice being less active. Further, these mice grow and mature normally in striking contrast to PC1 knock-out which is runted as a result of improper GHRH processing.

These observations indicate that mice with the PC1 mutant gene are able to differentially process prohormones, potentially with relevance to altered sensitivity to calcium and/or the sub-cellular compartment in which specific prohormones are processed. In accordance with these discoveries, the present invention provides novel PC1 mutant polypeptides and polynucleotides, as well as non-human animals that contain such mutant PC1 sequences. The claimed inventions can be valuable resources in which to fully understand prohormone and neuropeptide processing, specifically with relevance to the regulation of energy homeostasis. These could lead to better means for diagnosing, preventing, and treating metabolic disorders such as hyper(pro)insulinemia and obesity.

Before describing the present invention in detail, it is to be understood that this invention is not limited to particular devices or biological systems, which can, of course, vary. It is also to be understood that, unless otherwise indicated, the terminology used herein is for the purpose of describing particular embodiments only, and is not intended to be limiting. The following sections provide guidance for making and using the compositions of the invention, and for carrying out the methods of the invention.

### II. Definitions

As used in this specification and the appended claims, the singular forms “a”, “an” and “the” include plural referents unless the content clearly dictates otherwise. Thus, for example, reference to “a PC1 mutant” includes a combination of two or more PC1 mutants, and reference to “a non-human animal” includes more than one such animal.

The term “animal model” of the present invention includes transgenic animals harboring a heterologous PC1 gene, naturally occurring animals with genetic mutations in the PC1 gene, and non-transgenic animals that have been treated with one or more agents, or combinations thereof. Any of which may serve as experimental models for studying a disease, e.g., obesity. For example, a mouse model of the present invention can be a mouse that harbors a mutation in the PC1 gene as described herein.

“Acidic amino acid” refers to a hydrophilic amino acid having a side chain pK value of less than 7. Acidic amino acids typically have negatively charged side chains at physiological pH due to loss of a hydrogen ion. Genetically encoded acidic amino acids include Glu (E) and Asp (D).

“Basic amino acid” refers to a hydrophilic amino acid having a side chain pK value of greater than 7. Basic amino acids typically have positively charged side chains at physiological pH due to association with hydronium ion. Genetically encoded basic amino acids include His (H), Arg (R) and Lys (K).

An “endogenous” element is defined herein to mean naturally occurring in a cell. An endogenous PC1 polypeptide or polynucleotide is defined to mean a PC1 sequence that is endogenous to the cell.

An “expression vector” is a polynucleotide construct, generated recombinantly or synthetically, with a series of specified polynucleotide elements that permit transcription of a particular polynucleotide (e.g., one encoding a mutant PC1 polypeptide) in a host cell. The expression vector can be part of a plasmid, virus, or polynucleotide fragment. Typically, the expression vector includes a polynucleotide to be transcribed which is operably linked to a promoter.

A “heterologous sequence” or a “heterologous nucleic acid,” as used herein, is one that originates from a source foreign to the particular host cell, or, if from the same source, is modified from its original form. Thus, a heterologous gene in a host cell includes a gene that, although being endogenous to the particular host cell, has been modified.

The term “homologous” when referring to proteins and/or protein sequences indicates that they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology. Methods for determining sequence similarity percentages (e.g., BLASTP and BLASTN using default parameters) are described herein.

A “host cell” refers to a cell into which a heterologous polynucleotide (e.g., one encoding a mutant PC1) can be or has been introduced. The heterologous polynucleotide can be introduced into the cell by any means, e.g., electroporation, calcium phosphate precipitation, microinjection, transformation, viral infection, and/or the like. Typically, the host cells are capable of growth in culture and capable of expressing a desired protein or a polypeptide (e.g., a mutant PC1 polypeptide). The host cells encompass cells in in vitro culture as well as cells that are within a host animal. While the preferred host cells in in vitro culture of this invention are mammalian cells, other cells may be used, e.g., bacterial, yeast, or insect host cells. Host cells that are within an animal refer to those host cells which have been introduced into an animal or are endogenous to that animal, and are capable of expressing a desired polypeptide.

The term “identical” or “sequence identity” in the context of two nucleic acid sequences or amino acid sequences refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). The CLUSTAL program is well described by Higgins and Sharp (1988) Gene 73:237-244; Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment.

The term “substantially identical” nucleic acid or amino acid sequence means that a nucleic acid or amino acid sequence comprises a sequence that has at least 90% sequence identity or more, preferably at least 95%, more preferably at least 98% and most preferably at least 99%, compared to a reference sequence using any of the programs described in the art (preferably BLAST) using standard parameters. For example, the BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

An “isolated” polypeptide or polynucleotide means a polypeptide or a polynucleotide which has been identified and separated and/or recovered from a component of its natural environment. This definition specifically includes polypeptides or polynucleotides present in situ in a host cell wherein the host cell in its native form lacks the polypeptide or the polynucleotide. Ordinarily, however, isolated polypeptide or polynucleotide will be prepared by at least one purification step.

The term “nucleic acid” or “polynucleotide” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides.

The term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The term “polypeptide” is used interchangeably herein with the term “protein,” and refers to a polymer of amino acid residues, e.g., as typically found in proteins in nature.

“Prohormone convertase” enzymes are specialized proteinases having conserved active site domains which are substrate specific and cleave exclusively at certain sets of basic residues, preferably Lys-Arg, Arg-Arg, Lys-Lys and Arg-Lys. This type of proteinase is responsible for processing large precursor proteins, such as prorelaxin, to their biologically active form. Examples of prohormone convertases include mammalian PC1, PC2, PC4, and furin.

“Prohormone convertase 1 (PC1)” refers to the various endopeptidases that share structural and functional similarities to the human and mouse PC1 enzymes. PC1 is also termed PC3, SPC1, SPC3, and Pcsk1. Exemplary polynucleotide sequences encoding human and mouse PC1s are NM_{—}000439 (human) and NM_{—}013628 (mouse). Sequences encoding other PC1 species or variants are exemplified below.

“Prohormone convertase 1 mutant” refers to a prohormone convertase 1 that comprises amino acid substitution, deletions, and/or insertions at certain positions. Specific PC1 mutants of the present invention include PC1 polypeptides which have defective autocatalytic activity. The PC1 mutants can have a missense mutation in the conserved Asn222 residue (e.g., substitution by ASP or Glu residue) in the catalytic domain. Substituting amino acids in the mutant PC1 can be genetically encoded amino acid residues. They can also be naturally-occurring or synthetic analogs of the genetically encoded amino acid residues. PC1 mutants of the present invention also encompass polynucleotide sequences that encode such mutant PC1 polypeptides.

The term “subject” includes mammals, especially humans, as well as other non-human animals such as mouse, rat, and rabbit.

A “targeting vector” means a polynucleotide molecule that includes arms of homology, the nucleotide sequence or allele (located within or between the arms of homology), e.g., a point mutation, that is to be incorporated into the target gene (e.g., the PC1 gene), and one or more selectable markers.

The term “transgene” means a nucleic acid sequence (e.g., one encoding a mutant PC1 polypeptide of the invention) which has been introduced into a cell. A transgene could be partly or entirely heterologous, i.e., foreign, to the transgenic animal or cell into which it is introduced, or can be homologous to an endogenous gene of the transgenic animal or cell into which it is introduced. A transgene can also be present in a cell in the form of an episome. A transgene can include one or more transcriptional regulatory sequences and any other nucleic acid, such as 5′ UTR sequences, 3′ UTR sequences, or introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA.

A “variant” of a molecule such as a PC1 polypeptide is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

### III. Novel Prohormone Convertase 1 Mutants

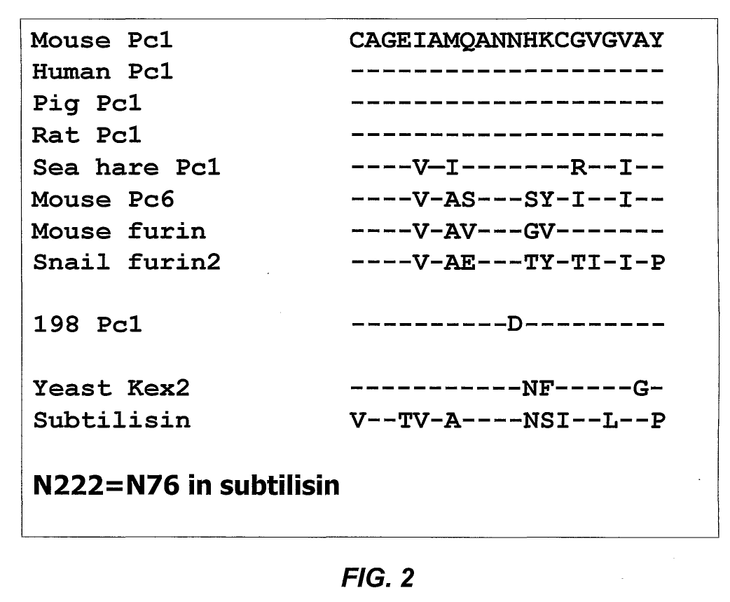

The present invention provides novel PC1 mutants that have some unique and useful biological and physiological properties. These PC1 mutants are novel and useful tools to study the mechanism by which PC1 mutations in particular, and peptide processing in general, lead to obesity. The PC1 mutants of the present invention typically contain a mutation in the catalytic domain which results in at least partially defective autocatalytic activity of the enzyme. The mutation is located in a peripheral loop in the catalytic domain that is responsible for binding calcium. In some embodiments, the mutation is a missense substitution in an asparagine residue corresponding to Asn²²² in the catalytic domain of mouse or human PC1. As shown in FIG. 2, Asn²²² is conserved in this family of serine proteases from bacteria to mammals, including mouse PC1, human PC1, pig PC1, rat PC1, furin, yeast Kex2 and bacterial substilisin. In some preferred embodiments, the PC1 mutants have the Asn²²² residue substituted with an acidic amino acid residue, e.g., Asp, Glu, or their analogs. In one embodiment, the Asn²²² residue is replaced with an aspartic acid residue.

This conserved Asn residue is located at amino acid position 222 in a number of PC1 proteins, e.g., human PC1 sequence NP_{—}000430 (Seidah et al., DNA Cell Biol. 11: 283-289, 1992; and Creemers et al., FEBS Lett. 300:82-88, 1992) or mouse PC1 sequence NP_{—}038656 (Seidah et al., Mol. Endocrinol. 5:111-122, 1991; and Seidah et al., DNA Cell Biol. 9:415-424, 1990). For this reason, this residue is referred to as Asn²²² herein regardless of which species the PC1 gene is from. However, it is understood that the precise position of this Asn-encoding codon in a given PC1 gene is not necessarily at position 222. Rather, the exact position of this conserved Asn residue in a PC1 polypeptide sequence can be easily determined by, e.g., sequence alignment with the human or mouse PC1.

As detailed in the Examples below, the PC1 mutants of the present invention have some unique and useful properties. Mice with a PC1^{N222D} mutant are found to be obese. The mutant mice have a weight more than three standard deviations above the mean of all mice screened, and a body fat content more than two standard deviations above the mean. These mice also had very high levels of insulin and/or proinsulin. The mutant mice also show a mild glucose intolerance probably due to the inability to secrete active insulin.

Biochemically, it appears that this mutation leads to a partial loss of function. Comparison of the mammalian PC1 sequence with the bacterial subtilisin sequence localizes the N222D mutation to a peripheral calcium binding loop. In vivo, the autocatalytic activity of the PC1 enzyme is impaired in tissues obtained from the N222D mice. Behaviorally, there appears to be no difference in food intake between the PC1 mutant and wildtype mice. Instead, the evidence is indicative of a difference in activity, and by extension in energy expenditure, between mutant and wildtype mice, the PC1 mutant mice being less active. Unlike PC1 knockout mice, PC1 N222D mice are not runted, thus potentially can process some prohormones (esp. GHRH) but not all. These PC1 mutants are useful for better understanding the cause of the obesity and identifying those prohormones which may be inefficiently processed by the mutant PC1 enzyme.

The PC1 mutants of the present invention can be easily produced from the wildtype PC1 sequences. Wildtype PC1 has been identified from various species. Sequences encoding these enzymes are also known in the art. Examples of polynucleotide sequences encoding various PC1 polypeptides or variants from the different species include NM_{—}000439 (homo sapiens), X64810 (human), NM_{—}174412 (Bos taurus), NM_{—}017091 (rat), M76705 (rat), NM_{—}013628 (mouse), M69196 (mouse), AB105175 (Rana catesbeiana), AF186405 (Bos taurus), AF092904 (Rana ridibunda), U40481 (Aplysia californica), U24128 (promoter and 5′ flanking region of human PC1 gene), and M58589 (mouse).

PC1 has also been structurally and functionally characterized. For example, cloning and sequencing of PC1 from various species has been described in the art, e.g., Seidah et al., DNA Cell Biol. 11: 283-289, 1992; Creemers et al., FEBS Lett. 300:82-88, 1992; Seidah et al., Mol. Endocrinol. 5:111-122, 1991; and Seidah et al., DNA Cell Biol. 9:415-424, 1990; Bloomquist et al., Cell. Physiol. Biochem. 3:197-212, 1993; Hakes et al., Endocrinol. 129: 3053-3063, 1991; Bloomquist et al., Mol. Endocrinol. 5:2014-2024, 1991; and Smeekens et al., Proc. Natl. Acad. Sci. USA 88, 340-344, 1991. Various other studies have further characterized the structural and biochemical properties of this enzyme, as described in, e.g., Li et al., Am. J. Physiol. Endocrinol. Metab. 280: E160-E170, 2001; Dey et al., J. Biol. Chem. 278: 15007-14, 2003; and Ueda et al., Proc. Natl. Acad. Sci. USA 100: 5622-27, 2003. The prohormone convertase enzymes are synthesized as inactive proenzymes, possessing an N-terminal signal peptide, prodomain, catalytic domain, P domain, and enzyme-specific C-terminal segment. The upstream prodomain acts as an internal chaperone for the folding of the catalytic domain in the endoplasmic reticulum (ER) and also as an intramolecular inhibitor. The activation of the enzymes requires autocatalytic cleavage and removal of the prodomain, a process that usually begins before they exit from the ER. The solution structure of the prodomain of PC1 was determined by heteronuclear NMR spectroscopy (Tangrea et al., J. Mol. Biol. 320: 801-821, 2002). The catalytic domain forms the core structure and contains the well-conserved catalytic triad Asp-His-Ser. This domain of the prohormone convertases has shown the greatest similarity (45%) in comparison of sequence alignments of all of the mammalian convertases and kexin. Molecular modeling studies have indicated that the catalytic domains of furin and PC1 are closely related to the bacterial subtilisins.

Any of the wildtype PC1 sequences or their fragments (e.g., a fragment containing the catalytic domain) can be employed to produce the mutant PC1 polynucleotides of the present invention. The mutant PC1 polynucleotides of the invention can be RNA, DNA, PNA or chimeras. They can be single-stranded, double stranded, or a mixed hybrid. Except for the mutated residues or regions, they are identical or substantially identical to any of the known wildtype PC1 sequences. Typically, they comprises at least 15, at least 25, at least 50, at least 100, at least 200, or at least 500 nucleotides surrounding the codon encoding the N222 residue. The invention also includes expression vectors, cell lines, and transgenic organisms comprising a mutant PC1 polynucleotide.

Polynucleotide sequences encoding the PC1 mutants of the present invention can be generated using methods well known and routinely practiced in the art, e.g., by site-directed mutagenesis of known wildtype PC1 sequences. Site-directed mutagenesis is a routinely practiced method for creating substitutions, deletions, and insertions at a target gene (e.g., a PC1 gene). This technique is well known in the art, e.g., as described in Adelman et al., DNA 2: 183, 1983. Briefly, a PC1 gene can be mutated by hybridizing an oligonucleotide encoding the desired mutation to a DNA template, where the template is the single-stranded form of a plasmid or bacterial phage containing the unaltered or native DNA sequence of the PC1 gene. After hybridization, a DNA polymerase is used to synthesize an entire second complementary strand of the template that will thus incorporate the oligonucleotide primer, and will code for the selected alteration in the PC1 gene. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. USA, 75: 5765, 1978).

Any of the residues in the peripheral loop in the catalytic domain wherein N222 is located can be the site for mutagenesis, provided that the mutagenesis leads to at least partial loss of autocatalytic activity of PC1. Partial loss of the autocatalytic activity of PC1 can be determined by monitoring the accumulation of a small PC1 polypeptide (e.g., 66 kD polypeptide for mouse PC1), as shown in the Examples below. In some preferred embodiments, the PC1 mutant polynucleotide sequences encode an acidic residue (e.g., Asp or Glu) at position 222 in the catalytic domain. For example, when a mouse PC1 polynucleotide sequence is employed, a mutant PC1 DNA sequence can contain GAT, GAC, GAA, or GAG at codon position 222 as opposed to an Asn-encoding codon, e.g., AAT.

Cells that harbor a mutant PC1 polynucleotide sequence described herein are also provided in the invention. Such cells can be produced, e.g., by generating the desired mutation in vitro and introducing the mutation into the genome of the cells by, e.g., homologous recombination. The desired mutation in a PC1 polynucleotide can be created by recombinant or synthetic means. Homologous recombination is a well-known and routinely practiced method for introducing specific mutations into the genome of a cell. The basic concept and procedures are described in more detail below.

Recombinant means for generating mutations in polynucleotide sequence are well known in the art. In addition to site-directed mutagenesis described above, other methods are also known in the art. See, e.g., Sambrook et al., Molecular Cloning: A Laboratory Manual, Cold Spring Harbor Press, N.Y., 3^{rd} Ed. (2000); Ausubel et al., Current Protocols in Molecular Biology, John Wiley & Sons, Inc., New York (1999); and Berger and Kimmel, (1987) Methods In Enzymology, Vol. 152: Guide To Molecular Cloning Techniques, San Diego: Academic Press, Inc. Alternatively, PC1 polynucleotides or fragments can be chemically synthesized using routine methods well known in the art (see, e.g., Narang et al., 1979, Meth. Enzymol. 68:90; Brown et al., 1979, Meth. Enzymol. 68:109; Beaucage et al., 1981, Tetra. Lett., 22:1859). In some embodiments, mutant PC1 polynucleotides of the invention contain non-naturally occurring bases, e.g., deoxyinosine (see, Batzer et al., 1991, Nucleic Acid Res. 19:5081; Ohtsuka et al., 1985, J. Biol. Chem. 260:2605-2608; Rossolini et al., 1994, Mol. Cell. Probes 8:91-98) or modified backbone residues or linkages.

### IV. PC1 Mutant Polypeptides

In addition to isolated polynucleotide sequences encoding the PC1 mutants, the present invention also provides isolated, substantially pure, or recombinant mutant PC1 polypeptides. As disclosed above, the mutant PC1 polypeptides contain a mutation in the catalytic domain which results in defective autocatalytic activity. Except for the mutated residue(s), the mutant PC1 polypeptides encompass polypeptides or proteins having an amino acid sequence that is identical or substantially identical to any of the known PC1 amino acid sequences (e.g., those described below). They also include polypeptides encoded by a mutant PC1 polynucleotide sequence described herein, their fragments that contain the mutation, allelic variants, and homologs thereof.

These PC1 mutant polypeptides can be obtained by recombinant production with a PC1 polynucleotide harboring the desired mutation (e.g., AAT to GAT replacement at the N222 codon position) described above. They can also be produced synthetically, utilizing the wildtype PC1 polypeptide sequences. Amino acid sequences encoding the various PC1 enzymes include NP_{—}000430 (Homo sapiens), NP_{—}776837 (Bos Taurus), NP_{—}058787 (Rattus norvegicus), NP_{—}038656 (Mus musculus), BAC98350 (Rana catesbeiana), P28840 (rat), P29120 (human), KXHUC1 (human), S59959 (pig), KXMSC1 (mouse), KXRTC1 (rat), and AAG17017 (Bos Taurus).

Vectors that express the mutant PC1 polynucleotide sequences are provided in the present invention. Typically, PC1 mutant polynucleotides of the invention are used in such expression vectors for the preparation of PC1 polypeptides. Expression vectors typically include transcriptional and/or translational control signals (e.g., the promoter, ribosome-binding site, and ATG initiation codon). In addition, the efficiency of expression can be enhanced by the inclusion of enhancers appropriate to the cell system in use. For example, the SV40 enhancer or CMV enhancer can be used to increase expression in mammalian host cells. Such expression vectors can be generated using methods that are well known and routinely practiced in the art, e.g., as described in Sambrook et al., supra; and Ausubel et al., supra.

The present invention also provides host cells that have incorporated an expression vector which expresses a mutant PC1 polypeptide described herein. In some embodiments, the expression vector encoding a PC1 polypeptide of the present invention is capable of being introduced into and expression in an in vitro host cell, such as a bacterial (e.g., E. coli, B. subtilus), yeast (e.g., Saccharomyces), insect (e.g., Spodoptera frugiperda), or mammalian cell culture systems. Examples of mammalian cell culture systems useful for expression and production of the PC1 polypeptides of the present invention include human embryonic kidney line 293 (Graham et al., 1977, J. Gen. Virol. 36:59); CHO (ATCC CCL 61 and CRL 9618); human cervical carcinoma cells (HeLa, ATCC CCL 2); and others known in the art. The use of mammalian tissue cell culture to express polypeptides is discussed generally in Winnacker, FROM GENES TO CLONES (VCH Publishers, N.Y., N.Y., 1987) and Ausubel et al., supra.

In some embodiments, promoters from mammalian genes or from mammalian viruses are used, e.g., for expression in mammalian cell lines. Suitable promoters can be constitutive, cell type-specific, stage-specific, and/or modulatable or regulatable (e.g., by hormones such as glucocorticoids). Useful promoters include, but are not limited to, the metallothionein promoter, the constitutive adenovirus major late promoter, the dexamethasone-inducible MMTV promoter, the SV40 promoter, and promoter-enhancer combinations known in the art.

The mutant PC1 polypeptides or fragments can also be expressed in transgenic animals (mouse, sheep, cow, etc.) and plants (tobacco, arabidopsis, etc.) using appropriate expression vectors which integrate into the host cell chromosome. Methods for generating transgenic animals, particularly animals such as mice or rats, have become conventional in the art and are described, e.g., in U.S. Pat. Nos. 4,736,866 and 4,870,009.

Except for the mutated residue or residues, the PC1 mutant polypeptides have amino acid sequences that are identical or substantially identical to any of the known wildtype PC1 sequences described above. In some embodiments, a mutant PC1 polypeptide has an amino acid sequence identical or substantially identical to the amino acid sequence encoded by a mutant PC1 polynucleotide nucleotide described above. In other embodiments, the mutant PC1 polypeptides are variants and mutants characterized by conservative substitutions of amino acid residues of the amino acid sequences encoded by a mutant PC1 polynucleotide sequence of the invention.

The mutant PC1 polypeptides of the invention may be a full-length PC1 polypeptide, or a fragment thereof which harbors the mutation in the catalytic domain. Typically, they contain at least 20, at least 40, at least 60 or at least 100 residues of the PC1 amino acid sequence spanning amino acid residue 222 in the catalytic region. Also provided by the invention are mutant PC1 polypeptides that are modified, relative to the amino acid sequence encoded by a mutant PC1 polynucleotide sequence described above, in some manner, e.g., truncated, mutated, derivatized, or fused to other sequences (e.g., to form a fusion protein). In addition to the mutation (e.g., N222 substitution in the catalytic domain) described above, some mutant PC1 polypeptides can contain additional mutations, e.g., insertions, deletions or substitutions of amino acid residues.

Typically, variants of the mutant PC1 polypeptides are structurally and functionally similar to a mutant PC1 polypeptide described herein. Structurally similarity is indicated by, e.g., substantial sequence identity, or immunological cross-reactivity. In some embodiments, a mutant PC1 polypeptide of the invention is a fusion protein or a fragment (e.g., the catalytic domain) of the full-length mutant PC1 polypeptide. In addition, a mutant PC1 polypeptide can be modified by substituting one or more amino acid residues with a D-amino acid of the same type (e.g., D-lysine in place of L-lysine) to generate more stable peptides. Similarly, modification of the amino or carboxyl terminals can also be used to confer stabilizing properties upon the polypeptides of the invention, e.g., amidation of the carboxyl-terminus or acylation of the amino-terminus or pegylated derivatives.

Suitable recombinant techniques for expressing mutant PC1 polypeptides from the mutant PC1 polynucleotides are disclosed herein. See also, Sambrook et al., supra; and Ausubel et al., supra. Synthetic methods that are suitable for synthesizing polypeptides such as the mutant PC1 polypeptides of the invention, variants, or fragments are described in Merrifield, 1963, Amer. Chem. Soc. 85:2149-2456, Atherton et al., 1989, SOLID PHASE PEPTIDE SYNTHESIS: A PRACTICAL APPROACH, IRL Press, and Merrifield, 1986, Science 232:341-347.

Isolation and purification of mutant PC1 polypeptides of the present invention can be carried out by methods that are generally well known in the art. These methods include, but are not limited to, ion exchange, hydrophobic interaction, HPLC or affinity chromatography, to achieve the desired purity. In one embodiment, mutant PC1 polypeptides are purified using immunoaffinity chromatography.

As an example, the present inventors have employed the baculovirus-based expression vectors and the Sf9 insect host cells to express mouse PC1 polypeptides. PC1 polypeptides thus expressed have been purified using conventional chromatography. The purified PC1 enzyme is useful in various applications, e.g., to determine optimal substrates of PC1 and its kinetics on synthetic substrates.

### V. Non-Human Animals Harboring Prohormone Convertase 1 Mutations

The present invention further provides non-human animals which harbor a mutant PC1 gene described above. These include animals with a mutation in the endogenous PC1 gene and transgenic animals which contain an exogenous PC1 mutant gene described herein. Non-human animals that are either homozygous or heterozygous for the mutation in the PC1 gene are included in the invention. Preferably, the animal is homozygous for the mutation.

Non-human animals harboring a mutant PC1 gene of the present invention can be generated using routinely practiced methods. In some embodiments, they can be obtained by screening mutagenized animals for metabolic abnormalities, and identifying PC1 mutations that lead to amino acid substitutions at N222 or a loss of the autocatalytic activity. For example, animals carrying various mutations can be generated by N-ethyl-N-nitrosurea (ENU) mediated mutagenesis. As shown in the Examples below, the present inventors have so obtained mice which harbor a PC1 mutant gene described herein.

Other than screening for PC1 mutants from mutagenized animals with metabolic abnormalities, the non-human animals of the present invention are more typically obtained by genetic manipulation. In some preferred embodiments of the invention, non-human animals harboring a desired PC1 mutant gene are created by genetic alteration of the wildtype PC1 gene in a non-human animal, e.g., by targeted mutagenesis. Such gene targeting methods have been well described in the art for generating non-human animals that contain specific gene mutations, e.g., U.S. Pat. No. 6,284,944. Briefly, the first step in producing a gene-targeted non-human mammal is to prepare a DNA targeting vector. The targeting vector is designed to replace, via homologous recombination, part of the endogenous PC1 gene sequence of a non-human mammal, so as to introduce the desired mutation (e.g., N222D substitution). The targeting vector is used to transfect non-human mammalian cell, e.g., a pluripotent, murine embryo-derived stem (“ES”) cell. In the cell, homologous recombination (i.e., the gene-targeting event) takes place between the targeting vector and the target gene. The mutant cell is then used to produce intact non-human mammals (e.g., by aggregation of murine ES cells to mouse embryos) to generate germ-line chimeras. The germline chimeras are used to produce siblings heterozygous for the mutated targeted gene. Finally, interbreeding of heterozygous siblings yields non-human mammals (e.g., mice) homozygous for the mutated target gene.

Targeting vectors for the practice of this invention can be constructed using materials, information and processes known in the art. A general description of the targeting vector used in this invention follows. A targeting vector or replacement vector for use in this invention has two essential functions: (1) to integrate specifically (and stably) at the endogenous PC1 target gene; and (2) to replace a portion of the endogenous PC1 gene, thereby introducing the desired mutation around the N222 residue. Those two essential functions depend on two basic structural features of the targeting vector.

The first basic structural feature of the targeting vector is a pair of regions, known as “arms of homology”, which are homologous to selected regions of the endogenous PC1 gene or regions flanking the PC1 gene. This homology causes at least part of the targeting vector to integrate into the chromosome, replacing part or all of the PC1 target gene, by homologous recombination.

The second basic structural feature of the targeting vector consists of the actual base changes (mutations) to be introduced into the target gene. In some embodiments, the base changes, e.g., AAT to GAT or GAA, in codon 222 of the catalytic domain of a PC1 gene (e.g., mouse or human PC1) results in the N222 residue being replaced with an Asp or a Glu residue. The mutation(s) to be introduced into the PC I target gene must be located within the “arms of homology.”

Gene targeting, which affects the structure of a specific endogenous gene in a cell, is to be distinguished from other forms of stable transformation, wherein integration of exogenous DNA for expression in a transformed cell is not site-specific, and thus does not predictably affect the structure of any particular gene already in the transformed cell. Furthermore, with the type of targeting vector preferred in the practice of this invention, a reciprocal exchange of genomic DNA takes place (between the “arms of homology” and the target gene), and chromosomal insertion of the entire vector is advantageously avoided.

Various targeting vectors can be employed to practice the present invention. For a given vector, the length of the arms of homology that flank the replacement sequence can vary considerably without significant effect on the practice of the invention. The arms of homology must be of sufficient length four effective heteroduplex formation between one strand of the targeting vector and one strand of a transfected cell's chromosome, at the PC1 target gene locus. The base pairs to be changed in the PC1 target gene must lie within the sequence that constitutes the arms of homology. The arms of homology may lie within the PC1 target gene, but it is not necessary that they do so and they may flank the PC1 target gene.

Preferably, the targeting vector will comprise, between the arms of homology, a positive selection marker. The positive selection marker should be placed within an intron of the target gene, so that it will be spliced out of mRNA and avoid the expression of a target/marker fusion protein. More preferably the targeting vector will comprise two selection markers; a positive selection marker, located between the arms of homology, and a negative selection marker, located outside the arms of homology. The negative selection marker is a means of identifying and eliminating clones in which the targeting vector has been integrated into the genome by random insertion instead of by homologous recombination. Exemplary positive selection markers are neomycin phosphotransferase and hygromycin β phosphotransferase genes. Exemplary negative selection markers are Herpes simplex thymidine kinase and diphtheria toxin genes.

To eliminate potential interference on expression of the target protein, the positive selection marker can be flanked by short loxP recombination sites isolated from bacteriophage P1 DNA. Recombination between the two loxP sites at the targeted gene locus can be induced by introduction of cre recombinase to the cells. This results in the elimination of the positive selection marker, leaving only one of the two short loxP sites (see, e.g., U.S. Pat. No. 4,959,317). Excision of the positive selectable marker from the mutated PC1 gene can thus be effected.

If base pair changes (mutations) are introduced into one of the arms of homology it is possible for these changes to be incorporated into the cellular gene as a result of homologous recombination. Whether or not the mutations are incorporated into cellular DNA as a result of homologous recombination depends on where the crossover event takes place in the arm of homology bearing the changes. For example, the crossover in the arm occurs proximal to the mutations and so they are not incorporated into cellular DNA. In another scenario, the crossover takes place distal to the position of the mutations and they are incorporated into cellular DNA. Because the location of the crossover event is random, the frequency of homologous recombination events that include the mutations is increased if they are placed closer to the positive selection marker.

By the above method, the skilled artisan can achieve the incorporation of the selectable marker at a preselected location in the PC1 target gene flanked by specific base pair changes. Presumably, the artisan would preferably choose to have the selectable marker incorporated within the intron of the target gene so as not to interfere with endogenous gene expression while the mutations would be included in adjacent coding sequence so as to make desired changes in the protein product of interest. See Askew et al., Mol Cell Biol 13: 4115-4124, 1993; Fiering et al., Proc. Natl. Acad. Sci. USA 90: 8469-8473, 1993; Rubinstein et al., Nuc. Acid Res. 21: 2613-2617, 1993; Gu et al., Cell 73: 1155-1164, 1993; and Gu, et al., Science 265: 103-106, 1994).

In addition to non-human animals which have its endogenous PC1 gene mutated in accordance with the present invention, transgenic animals harboring a heterologous PC1 mutant gene are also included in the present invention. For example, a transgenic non-human animal of the present invention can be a mouse which contains a transgene which encodes a human PC1 mutant gene. Typically, such transgenic animals have their endogenous PC1 gene replaced with the PC1 mutant transgene.

Transgenic animals (e.g., transgenic mice) expressing a mutant PC1 gene from a different species (e.g., human) can be generated according to methods well known in the art. For example, techniques routinely used to create and screen for transgenic animals have been described in, e.g., see Bijvoet et al., Hum. Mol. Genet. 7:53-62, 1998; Moreadith et al., J. Mol. Med. 75:208-216, 1997; Tojo et al., Cytotechnology 19:161-165, 1995; Mudgett et al., Methods Mol. Biol. 48:167-184, 1995; Longo et al., Transgenic Res. 6:321-328, 1997; U.S. Pat. Nos. 5,616,491; 5,464,764; 5,631,153; 5,487,992; 5,627,059; 5,272,071; and WO 91/09955, WO 93/09222, WO 96/29411, WO 95/31560, and WO 91/12650. Methods for generating transgenic non-human animals expressing a mutant human gene are also taught in the art, e.g., U.S. Pat. No. 6,284,944.

### VI. Genetic Screening for Predisposition to Developing Obesity

The present invention provides novel methods of genetic screening for identifying a predisposition to developing obesity. These methods can be used alone or in combination with other known methods of screening for genetic causes of obesity. The methods involve identifying in a subject specific prohormone convertase 1 alleles which are associated with obesity, based on the discovery that obesity can be caused by mutations surrounding the N222 residue in the PC1 gene. Typically, the mutations result in defective autocatalytic activity of the encoded PC1 polypeptide.

The methods can be used to identify either homozygous PC1 mutation or a heterozygous carrier state. In general, the methods involve preparing a nucleic acid sample from the subject for screening and then assaying the sample for one or more of the mutant PC1 alleles. In some embodiments, the methods comprise the steps of (a) analyzing the PC1 nucleic acid in a sample obtained from a subject, and (b) determining the presence of a mutation at codon position 222 (e.g., one which corresponds to a N222D or N222E missense mutation) in the subject. The presence of such a mutation is indicative of a risk of or a propensity to developing obesity.

The nucleic acid sample from the subject contains a nucleic acid sequence correlating to the PC1 gene. It can be in the form of genomic DNA, an mRNA or cDNA, and in single or double stranded form. Preferably, genomic DNA is used because of its relative stability in biological samples compared to mRNA. The sample can be obtained from any cells that contain a PC1 gene. If appropriate, the sample can be obtained from tissues in the subject where PC1 is normally expressed. PC1 is known to be expressed in the brain and hypothalamus. Other neuroendocrine tissues where PC1 is expressed include pancreas (B cells and a cells), intestine (e.g., L cells), and pituitary.

When mRNA from a sample from the subject is used, the cells will be lysed under RNase inhibiting conditions. Typically, the first step is to isolate the total cellular mRNA. Poly A+ mRNA can then be selected (e.g., by hybridization to an oligo-dT cellulose column). Preferably, the nucleic acid sample is enriched for a presence of PC1 allelic material. For example, enrichment can be accomplished by subjecting the genomic DNA or mRNA to a polymerase chain reaction (PCR) to produce an amplified PCR product. PCR amplification methods are well known in the art and described in detail, e.g., in U.S. Pat. Nos. 4,683,192, 4,683,202, 4,800,159, and 4,965,188. Additional teachings of PCR are provided in, e.g., Sambrook et al., supra; Ausubel et al., supra; PCR Technology: Principles and Applications for DNA Amplification, Erlich (ed.), Stockton Press, New York (1989); and PCR Protocols: A Guide to Methods and Applications, Innis et al. (eds.), Academic Press, San Diego, Calif. (1990).

After amplification of the nucleic acid sample from the subject, sequence analysis is then performed. Unknown single-nucleotide substitutions or other sequence differences in the PC1 target polynucleotide sequence can be detected with standard molecular biology methods. Such methods are described in the art, e.g., U.S. Pat. No. 4,946,773, Sambrook et al., supra; and Ausubel et al., supra.

### EXAMPLES

The following examples are offered to illustrate, but not to limit the claimed invention.

### Example 1

### Mice with a PC1^{N222D} Mutation are Obese

This Example describes mice with a PC1^{N222D} mutation and their obesity phenotype.

In a forward genetic screen of ENU-mutagenized mice for metabolic abnormalities, we identified two mice in the same family that were obese. These mice have a weight more than three standard deviations above the mean of all mice screened, and a body fat content more than two standard deviations above the mean. These mice also had very high levels of insulin and/or proinsulin (z-score ≧3). Sequencing of the candidate genes listed at the lower right revealed a mutation in the prohormone convertase 1 (PC1) gene. The mutation is a G to an A in the coding sequence leading to an amino acid substitution of aspartic acid for asparagine at position 222 (N222D). One of these founder mice (10609) is homozygous for this mutation while the other (10875) is heterozygous.

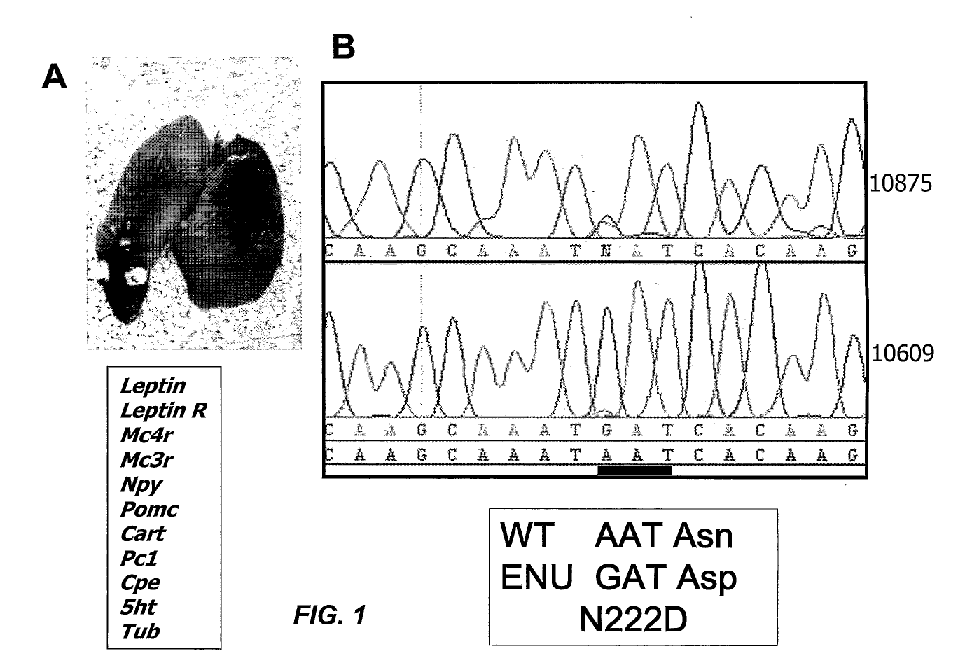

The mouse homozygous for this mutation, 198(10609) PC1, is pictured in FIG. 1A (the mouse on the right), next to a wildtype mouse (the mouse on the left). FIG. 1B shows sequencing chromatogram of PC1 exon 7 for the two founder obese mice and wildtype PC1 from NCBI. 10875 and 10609 in the figure refer to the two different founders from this family.

The N222 residue of mouse PC1 corresponds to N76 of bacterial subtilisin. As shown in FIG. 2, the N222 residue of mouse PC1 is at a position that is conserved in this family of serine proteases from bacteria to mammals. The Asn to Asp mutation leads to slight structural change with gain of negative charge. Based on crystal structure of bacterial subtilisin and a comparison of the mammalian PC1 sequence with the bacterial sequence, it is clear that the N222D mutation is located in a peripheral calcium binding loop in the catalytic domain.

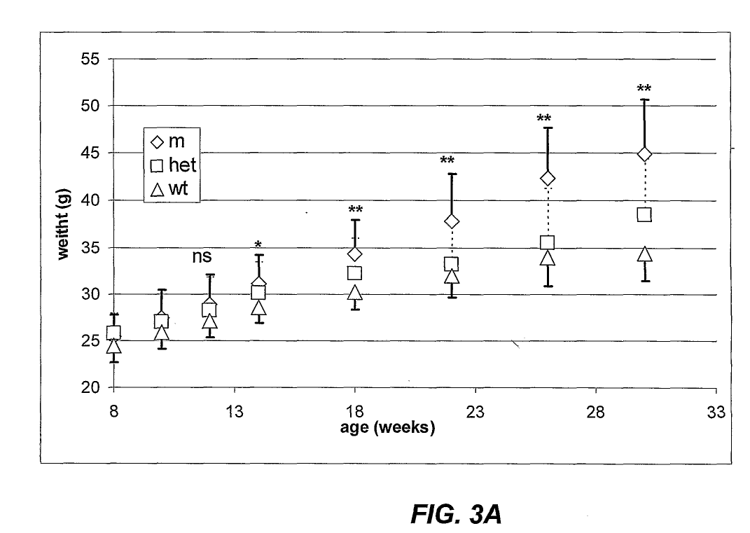

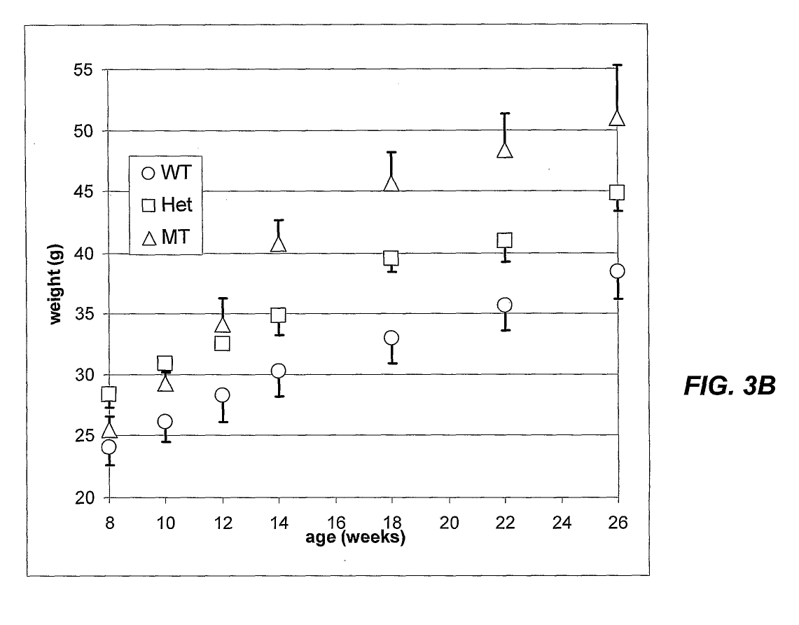

FIG. 3A shows the weight of wildtype and mutant PC1 mice on normal chow. The PC1 mutant mice and their heterozygous or wildtype littermates were fed a normal chow diet (11% calories from fat), and their weight was measured periodically. Mutant mice show significantly higher weight by twelve weeks of age and are 30% heavier at 30 weeks. [*—p<0.05, m vs. wt, student's t-test. **—p<0.005, m vs. wt, student's t-test.]

FIG. 3B shows the weight of wildtype and mutant PC1 mice on breeder's diet (21% calories from fat). The PC1 homozygous mutant mouse and their heterozygous or wildtype littermates were fed a breeder's diet (21% calories from fat) and their weight was measured periodically.

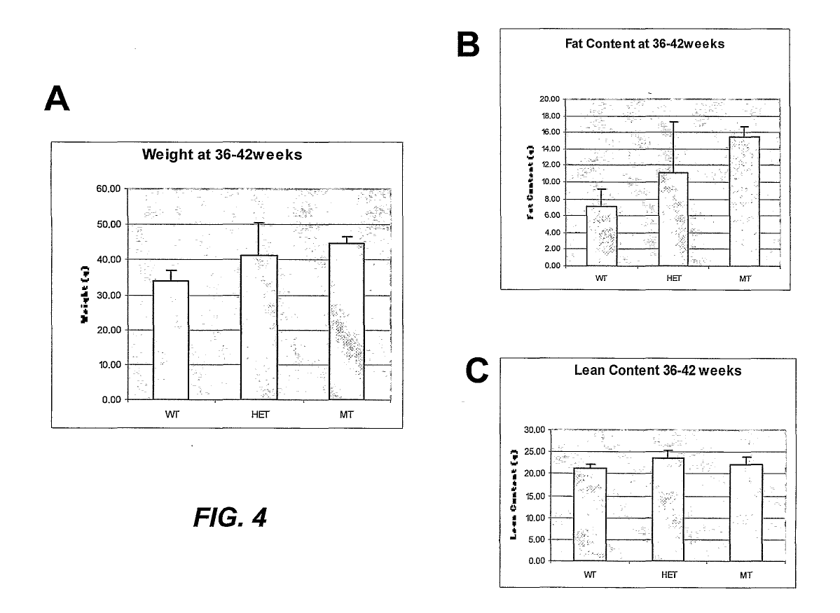

Body composition of the mutant mouse was also examined, using an Echo Medical Systems (Houston, Tex.) whole body composition analyzer. The results are shown in FIGS. 4A-4C. Compared to the wildtype littermate, the PC1^{N222D} mutant mouse has higher weight (FIG. 4A) and significantly higher fat content (FIG. 4B). However, the lean content of the mutant mouse is similar to that of the wildtype mouse (FIG. 4C).

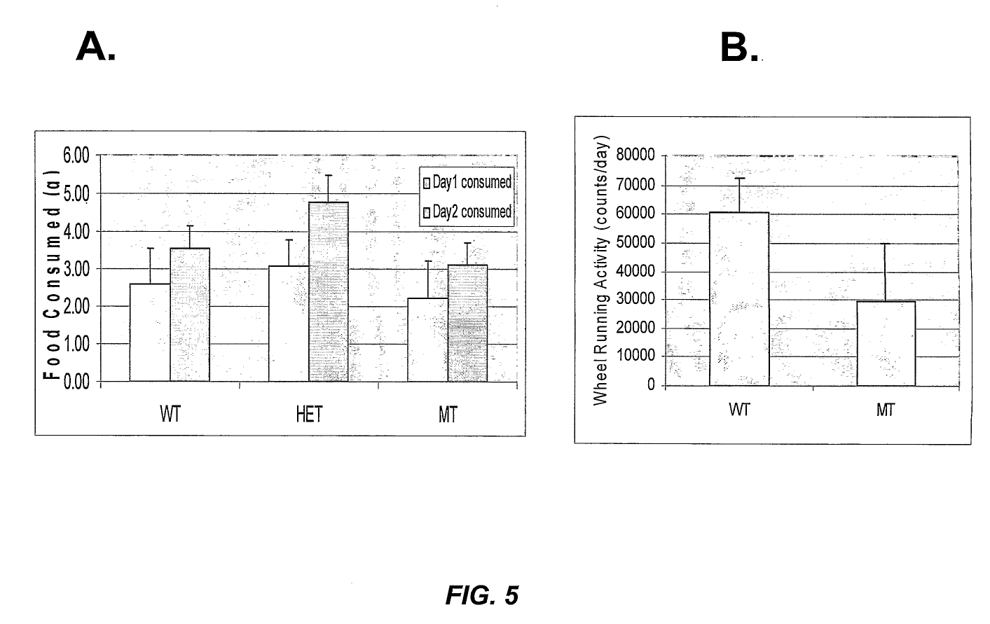

Further analysis indicates that there is no significant difference in food consumption between the wildtype and the PC1^{N222D} mutant mouse (FIG. 5A). Instead, the difference between the wildtype and mutant mouse appears to be in the activity, and presumably in energy expenditure (FIG. 5B).

It was previously shown that mice with PC1 knockout are not obese. In fact they are normal at birth, and become runted due to severely growth impairment. This was attributed to a presumed inability to process pro-growth hormone releasing hormone (proGHRE). See Zhu et al. Proc Natl Acad Sci USA 99: 10293-10298, 2002. In contrast, the PC1^{N222D} mouse is not runted, thus potentially can process some prohormones (esp. GHRH) but not all. Thus, such mutant PC1 mice offer a unique opportunity to study the role of PC1 and peptide processing in the regulation of energy homeostasis.

### Example 2

### Elevated Insulin/Proinsulin Levels and Glucose Intolerance in PC1^{N222D} Mice

This Example describes hyper(pro)insulinemia, leptin resistance, and glucose intolerance in mice with the PC1^{N222D} mutation.

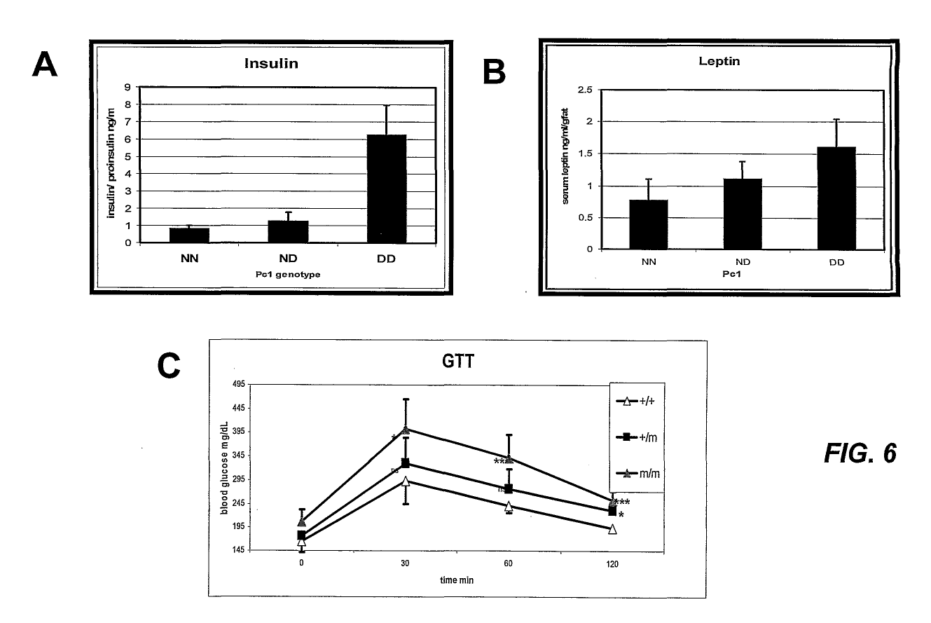

First, leptin and insulin levels in the mutant mouse were examined. The results were shown in FIG. 6. The Figure shows insulin/proinsulin (FIG. 6A) or leptin (FIG. 6B) levels in the PC1 homozygous mutant mouse or their heterozygous and wildtype littermates as assayed by ELISA. The insulin ELISA does not distinguish between insulin and proinsulin. The assays were done at ˜36 weeks of age. Elevation of insulin/proinsulin can be seen at a much earlier age, <18 weeks. The high levels of insulin/proinsulin in the mutant animals probably reflect their inability to process proinsulin to its active form. The mice then compensate for the reduced insulin function by secreting more insulin/proinsulin. Results from further insulin tolerance tests in wildtype, heterozygous, and mutant PC1 mice indicate that the PC1 mutant mice have increased insulin sensitivity, as demonstrated by an increased glucose-lowering effect of a fixed dose of insulin.

FIG. 6C shows results of a glucose tolerance test (GTT) performed on the PC1 mutant mice or their heterozygous or mutant littermates. The mutant mouse shows a mild glucose intolerance probably due to the inability to secrete active insulin. Male mice between 12 and 18 weeks old were injected ip with 1.5 mg/kg glucose. The tails were clipped and blood was drawn at the indicated times. Glucose concentration was determined with a glucometer (NN—wildtype mice. ND—mice heterozygous for the N222D mutation in the PC1 gene. DD—mice homozygous for the N222D mutation in the PC1 gene). The results indicate that PC1 mutant animals cannot dispose of a glucose load as well as wildtype animals. From these tests and the high insulin immunoreactivity in the plasma, it appears that the PC1 mutant mice are glucose intolerant due to the secretion of a partially active form of insulin, like proinsulin.

### Examples 3

### Defective PC1 Autocatalytic Activity of PC1^{N222D} Mice

This Example describes impaired autocatalytic processing of PC1 in the PC1^{N222D} mutant mice.

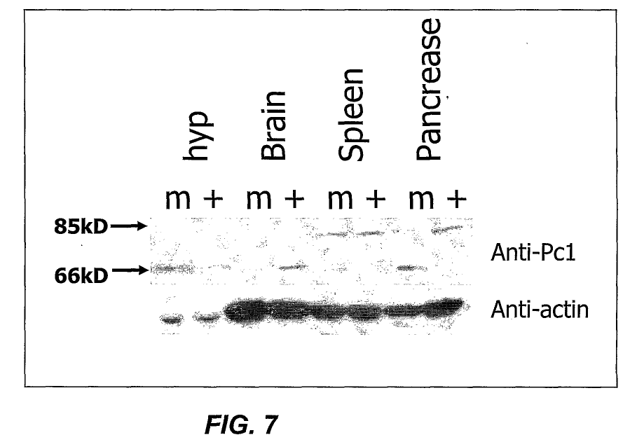

PC1 undergoes autocatalytic processing to its most active form of 66 kD. As shown in FIG. 7, this processing to the smaller form is defective in the PC1^{N222D} mutant mice, especially in brain and hypothalamus. In the brain and hypothalamus of wildtype mice, both the 85 kD and autocatalytically processed 66 kD form of PC1 are present. In mutant mice however, the processed 66 kD form is not detectable. The symbol “+” in the figure denotes tissue from wildtype mice, and “m” indicates tissue from homozygous mutant N222D mice.

The mutant PC1 can be used to study the mechanism by which PC1 mutations in particular, and peptide processing in general, lead to obesity. Since they are not runted, the PC1^{N222D} mutant mice presumably have normal activities in processing proGHRH to its mature form. The defective autocatalytic activity suggests that some other substrates of PC1 might be implicated in obesity. A likely candidate is α-MSH, which is involved in controlling appetite. Further studies indicate that the level of α-MSH is reduced in the hypothalami of PC1 mutant animals while plasma thyroid hormone is normal.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, patents, patent applications, accession numbers and their corresponding polynucleotide and polypeptide sequences, and/or other documents cited in this application are incorporated by reference in their entirety and for all purposes to the same extent as if each individual publication, patent, patent application, or other document were individually so denoted.

We claim:

1. An isolated mutant prohormone convertase 1 (PC1) polypeptide comprising a missense mutation at residue Asn²²² in the catalytic domain.

2. The mutant prohormone convertase 1 of claim 1, wherein the missense mutation is substitution of Asn²²² with an acidic amino acid residue.

3. The mutant prohormone convertase 1 of claim 2, wherein the acidic amino acid residue is aspartic acid.

4. The mutant prohormone convertase 1 of claim 1, wherein the prohormone convertase 1 is from a mammalian species.

5. The mutant prohormone convertase 1 of claim 4, wherein the prohormone convertase 1 is from human.

6. The mutant prohormone convertase 1 of claim 5, wherein the human prohormone convertase has an amino acid sequence of accession number NP_{—}000430, P29120, or KXHUC1.

7. The mutant prohormone convertase 1 of claim 4, wherein the prohormone convertase 1 is from mouse.

8. The mutant prohormone convertase 1 of claim 7, wherein the mouse prohormone convertase has an amino acid sequence of accession number NP_{—}038656 or KXMSC1.

9. An isolated polynucleotide encoding a mutant prohormone convertase 1 (PC1) polypeptide, wherein the mutant prohormone convertase polypeptide comprises a missense mutation at residue Asn²²² in the catalytic domain.

10. The polynucleotide of claim 9, wherein the missense mutation is substitution of Asn²²² with an acidic amino acid residue.

11. The polynucleotide of claim 10, wherein the acidic amino acid residue is aspartic acid.

12. The polynucleotide of claim 9, wherein the prohormone convertase 1 is from a mammalian species.

13. The polynucleotide of claim 12, wherein the prohormone convertase 1 is from human.

14. The polynucleotide of claim 13, wherein the human prohormone convertase 1 is encoded by a polynucleotide sequence having accession number NM_{—}000439 or X64810.

15. The polynucleotide of claim 12, wherein the prohormone convertase 1 is from mouse.

16. The polynucleotide of claim 15, wherein the mouse prohormone convertase 1 is encoded by a polynucleotide sequence having accession number NM_{—}013628, M69196, or M58589.

17. A non-human animal which comprises a mutant prohormone convertase 1 (PC1) gene encoding a mutant prohormone convertase 1 polypeptide, wherein the mutant prohormone convertase 1 polypeptide comprises a mis sense mutation at residue Asn²²² in the catalytic domain.

18. The non-human animal of claim 17, wherein the missense mutation is substitution of Asn²²² with an acidic amino acid residue.

19. The non-human animal of claim 18, wherein the acidic amino acid residue is aspartic acid.

20. The non-human animal of claim 17, wherein the PC1 gene is from a mammalian species.

21. The non-human animal of claim 20 which is a mouse, wherein the PC1 gene is mouse PC1 gene.

22. The non-human animal of claim 20 which is a rat, wherein the PC1 gene is rat PC1 gene.

23. A non-human animal comprising, in its genome, a DNA sequence encoding a mutant PC1 polypeptide that is defective in its autocatalytic activity relative to wildtype form of the PC1 polypeptide.

24. The non-human animal of claim 23, wherein said mutant PC1 polypeptide comprises a missense mutation at residue N222.

25. The non-human animal of claim 24, wherein the missense mutation is substitution of N222 with an acidic residue.

26. The non-human animal of claim 25, wherein the acidic residue is Asp.

27. The non-human animal of claim 23, wherein the mutant PC1 polypeptide is mouse PC1 polypeptide.

28. The non-human animal of claim 23, wherein the mutant PC1 polypeptide is encoded by a transgene heterologous to the animal.
